(19) United States
(12) Patent Application Publication
(10) Pub. No.: US 2014/0316294 A1
(43) Pub. Date: Oct. 23, 2014

(54) METHOD AND APPARATUS FOR SUPPRESSING FAR-FIELD SENSING DURING ATRIAL MAPPING

(71) Applicant: Boston Scientific Scimed, Inc., Maple Grove, MN (US)

(72) Inventors: Barun Maskara, Blaine, MN (US); Shantha Arcot-Krishnamurthy, Vadnais Heights, MN (US); Pramodsingh H. Thakur, White Bear Lake, MN (US); Allan C. Shuros, St. Paul, MN (US); Sunipa Saha, Shoreview, MN (US); Shibaji Shome, Arden Hills, MN (US)

(73) Assignee: Boston Scientific Scimed, Inc., Maple Grove, MN (US)

(21) Appl. No.: 14/258,867

(22) Filed: Apr. 22, 2014

Related U.S. Application Data
(60) Provisional application No. 61/814,656, filed on Apr. 22, 2013.

(51) Int. Cl. A61B 5/0452

(52) U.S. Cl. CPC A61B 5/04525
USPC 600/509

(57) ABSTRACT

A method and system for mapping an anatomical structure includes sensing activation signals of intrinsic physiological activity with a plurality of electrodes disposed in or near the anatomical structure. Substantially similar activation signals are binned according to a self-correlation algorithm which identifies patterns among the sensed activation signals. A template is generated for each bin and compared to a characteristic template to identify at least one bin which corresponds to a far-field activation signal.

### CROSS-REFERENCE TO RELATED APPLICATION

This application claims priority to Provisional Application No. 61/814,656, filed Apr. 22, 2013, which is herein incorporated by reference in its entirety.

### TECHNICAL FIELD

The present disclosure relates to cardiac mapping systems. More specifically, the present disclosure relates to a cardiac mapping system configured to suppress far-field activation during mapping based on activation signals sensed by non-contact electrodes.

### BACKGROUND

Diagnosing and treating heart rhythm disorders often involve the introduction of a catheter having a plurality of sensors/probes into a cardiac chamber through the surrounding vasculature. The sensors detect electric activity of the heart at sensor locations in the heart. The electric activity is generally processed into electrogram signals that represent signal propagation through cardiac tissue at the sensor locations.

The sensors in a cardiac chamber may detect far-field electrical activity, i.e. ambient electrical activity away from the sensors, which can negatively affect the detection of local electrical activity, signals at or near the sensor location. For example, ventricular activation may present itself as far-field signals substantially simultaneously on multiple sensors situated in the atrium. Due to the magnitude of ventricular activations, the phenomenon can mask significant aspects of highly localized activity and thus result in inaccurate activation maps and/or reduced resolution activation maps upon which physicians rely to administer therapy, e.g. ablation therapy, to a patient.

### SUMMARY

In Example 1, a method for mapping an anatomical structure includes sensing activation signals of intrinsic physiological activity with a plurality of electrodes disposed in or near the anatomical structure, binning substantially similar sensed activation signals according to a self-correlation algorithm which identifies patterns among the sensed activation signals, and identifying at least one bin which corresponds to a far-field activation signal.

In Example 2, the method according to Example 1, wherein the step of identifying at least one bin further includes acquiring a plurality of far-field activation signals with at least one far-field sensor, aligning the far-field activation signals according to a characteristic feature, determining a temporal window for a far-field activation signal, and identifying at least one bin of activation signals which correspond to the temporal window as far-field activation signals.

In Example 3, the method according to either of Examples 1 and 2, wherein the step of identifying at least one bin further includes, generating a characteristic template for each bin based on a morphology of the corresponding activation signals, generating a morphology template which identifies a morphology of a far-field activation signal, and identifying at least one bin of activation signals as far-field activation signals according to a comparison of each characteristic template and the morphology template.

In Example 4, the method according to any of Examples 1-3, wherein the step of identifying at least one bin further includes generating a characteristic template for each bin based on a frequency component of the corresponding activation signals, generating a frequency template which identifies frequency components of a far-field activation signal, and identifying at least one bin of activation signals as far-field activation signals according to a comparison of each characteristic template and the frequency template.

In Example 4, the method according to any of Examples 1-4, wherein the step of identifying at least one bin further includes generating a characteristic template for each bin based on a temporal frequency of the corresponding activation signals, generating a temporal template which identifies a temporal frequency of a far-field activation signal, and identifying at least one bin of activation signals as far-field activation signals according to a comparison of each characteristic template and the temporal template.

In Example 5, the method according to any of Examples 1-4, further includes filtering the activation signals which correspond to the at least one identified bin from the sensed activation signals.

In Example 6, the method according to any of Examples 1-5, wherein the activation signals are filtered based on at least one of a subtraction between the activation signals and a template, a suppression of an amplitude for a duration of a beat, and zeroing the zeroing the signal for a duration of the beat.

In Example 7, the method according to any of Examples 1-6, further includes generating an activation map of the anatomical structure based on the filtered activation signals.

In Example 8, a method for mapping an anatomical structure includes sensing activation signals of intrinsic physiological activity with a plurality of electrodes disposed in or near the anatomical structure, binning substantially similar sensed activation signals according to a self-correlation algorithm which identifies patterns among the sensed activation signals, identifying at least one bin which corresponds to a far-field activation signal, filtering the activation signals which correspond to the at least one identified bin from the sensed activation signals; and generating an activation map of the anatomical structure based on the filtered activation signals.

In Example 9, the method according to Example 8, wherein the step of identifying at least one bin further includes acquiring a plurality of far-field activation signals with at least one far-field sensor, aligning the far-field activation signals according to a characteristic feature, determining a temporal window for a far-field activation signal, and identifying at least one bin of activation signals which correspond to the temporal window as far-field activation signals.

In Example 10, the method according to either Examples 8 and 9, wherein the step of identifying at least one bin further includes generating a characteristic template for each bin based on a morphology of the corresponding activation signals, generating a morphology template which identifies a morphology of a far-field activation signal; and identifying at least one bin of activation signals as far-field activation signals according to a comparison of each characteristic template and the morphology template.

In Example 11, the method according to any of Examples 8-10, wherein the step of identifying at least one bin further includes generating a characteristic template for each bin based on a frequency component of the corresponding activation signals, generating a frequency template which identifies frequency component of a far-field activation signal, and identifying at least one bin of activation signals as far-field activation signals according to a comparison of each characteristic template and the frequency template.

In Example 12, the method according to any of Examples 8-11, wherein the step of identifying at least one bin further includes generating a characteristic template for each bin based on a temporal frequency of the corresponding activation signals, generating a temporal template which identifies a temporal frequency of a far-field activation signal, and identifying at least one bin of activation signals as far-field activation signals according to a comparison of each characteristic template and the temporal template.

In Example 13, an anatomical mapping system includes a plurality of mapping electrodes configured to detect activation signals of intrinsic physiological activity within an anatomical structure, each of the plurality of mapping electrodes having an electrode location and channel, and a processing system associated with the plurality of mapping electrodes, the processing system configured to record the detected activation signals and associate at least one of the plurality of mapping electrodes with each recorded activation signal, the processing system further configured to bin substantially similar sensed activation signals according to a self-correlation algorithm which identifies patterns among the sensed activation signals, and identify at least one bin which corresponds to a far-field activation signal.

In Example 14, the anatomical mapping system according to Example 13, wherein the processing system is further configured to filter the activation signals which correspond to the at least one identified bin from the sensed activation signals, and generate an activation map of the anatomical structure based on the filtered activation signals.

In Example 15, the anatomical mapping system according to either of Examples 13 and 14, further includes a display configured to display the activation map of the filtered activation signals.

In Example 16, the anatomical mapping system according to any of Examples 13-15, wherein, to identify at least one bin which corresponds to a far-field activation signal, the processing system is further configured to align a plurality of far-field activation signals acquired with at least one far-field sensor according to a characteristic feature, determining a temporal window for a far-field activation signal, and identify at least one bin of activation signals which correspond to the temporal window as far-field activation signals.

In Example 17, the anatomical mapping system according to any of Examples 13-16, wherein, to identify at least one bin which corresponds to a far-field activation signal, the processing system is further configured to generate a characteristic template for each bin based on a morphology of the corresponding activation signals, generate a morphology template which identifies a morphology of a far-field activation signal, and identify at least one bin of activation signals as far-field activation signals according to a comparison of each characteristic template and the morphology template.

In Example 18, the anatomical mapping system according to any of Examples 13-17, wherein, to identify at least one bin which corresponds to a far-field activation signal, the processing system is further configured to generate a characteristic template for each bin based on a frequency component of the corresponding activation signals, generate a frequency template which identifies frequency component of a far-field activation signal, and identify at least one bin of activation signals as far-field activation signals according to a comparison of each characteristic template and the frequency template.

In Example 19, the anatomical mapping system according to any of Examples 13-18, wherein, to identify at least one bin which corresponds to a far-field activation signal, the processing system is further configured to generate a characteristic template for each bin based on a frequency component of the corresponding activation signals, generate a frequency template which identifies frequency component of a far-field activation signal, and identify at least one bin of activation signals as far-field activation signals according to a comparison of each characteristic template and the frequency template.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 a schematic view of an embodiment of a catheter system for accessing a targeted tissue region in the body for diagnostic and therapeutic purposes.

FIG. 2 is a schematic view of an embodiment of a mapping catheter having a basket functional element carrying structure for use in association with the system of FIG. 1.

FIG. 3 is a schematic side view of an embodiment of the basket functional element including a plurality of mapping electrodes.

FIG. 4 is a flow chart of a method for mapping an anatomical structure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

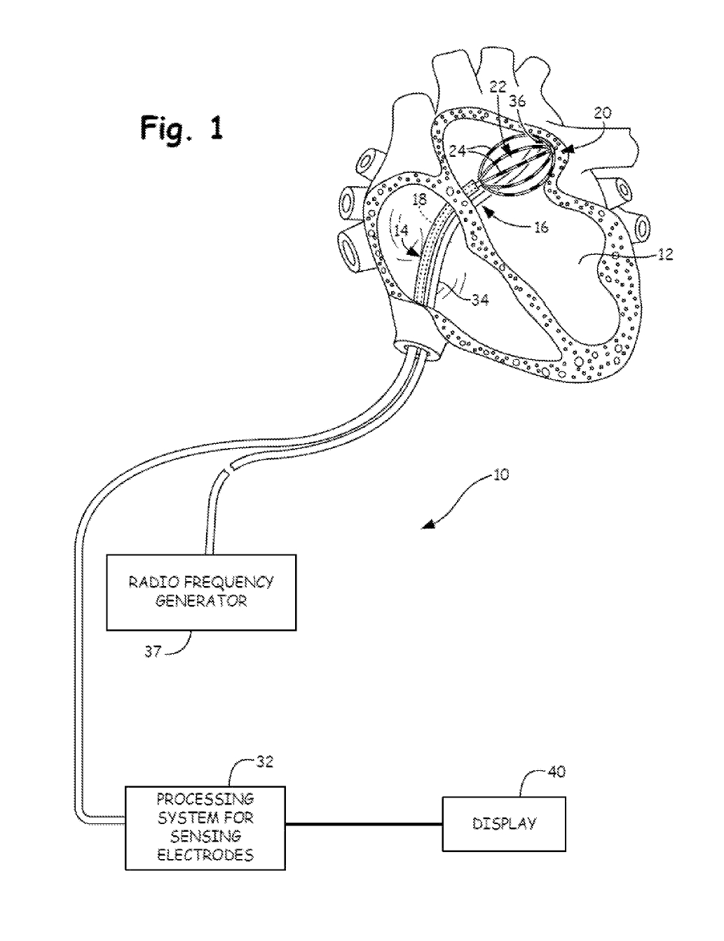

FIG. 1 is a schematic view of a system 10 for accessing a targeted tissue region in the body for diagnostic or therapeutic purposes. FIG. 1 generally shows the system 10 deployed in the left ventricle of the heart. Alternatively, system 10 can be deployed in other regions of the heart, such as the left atrium, right atrium, or right ventricle. While the illustrated embodiment shows the system 10 being used for ablating myocardial tissue, the system 10 (and the methods described herein) may alternatively be configured for use in other tissue ablation applications, such as procedures for ablating tissue in the prostrate, brain, gall bladder, uterus, and other regions of the body, including in systems that are not necessarily catheter-based.

The system 10 includes a mapping probe 14 and an ablation probe 16. In FIG. 1, each is separately introduced into the selected heart region 12 through a vein or artery (e.g., the femoral vein or artery) through suitable percutaneous access. Alternatively, the mapping probe 14 and ablation probe 16 can be assembled in an integrated structure for simultaneous introduction and deployment in the heart region 12.

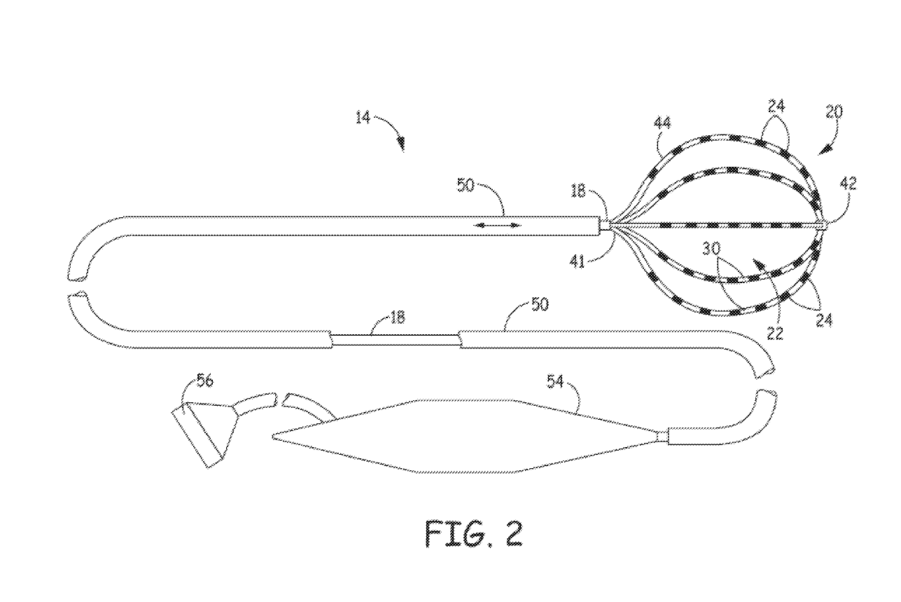

The mapping probe 14 has a flexible catheter body 18. The distal end of the catheter body 18 carries a three-dimensional multiple electrode structure 20. In the illustrated embodiment, the structure 20 takes the form of a basket defining an open interior space 22 (see FIG. 2), although other multiple electrode structures could be used wherein the geometry of the electrode structure and electrode locations are known. The multiple electrode structure 20 carries a plurality of mapping electrodes 24 each having an electrode location and channel. Each electrode 24 is configured to sense intrinsic physiological activity in the anatomical region on which the ablation procedure is to be performed. In some embodiments, the electrodes 24 are configured to detect activation signals of the intrinsic physiological activity within the anatomical structure, e.g., the activation times of cardiac activity.

The electrodes 24 are electrically coupled to a processing system 32. A signal wire (not shown) is electrically coupled to each electrode 24 on the basket structure 20. The wires extend through the body 18 of the probe 14 and electrically couple each electrode 24 to an input of the processing system 32, as will be described later in greater detail. The electrodes 24 sense intrinsic electrical activity in the anatomical region, e.g., myocardial tissue. The sensed activity, e.g. activation signals, is processed by the processing system 32 to assist the physician by generating an anatomical map, e.g., action potential duration (APD) map or an activation map, to identify the site or sites within the heart appropriate for ablation. The processing system 32 identifies a near-field signal component, i.e. activation signals associated with local activation and originating from the tissue adjacent to the mapping electrode 24, from an obstructive far-field signal component, i.e. activation signals originating from non-adjacent tissue, within the sensed activation signals. For example, in an atrial study, the near-field signal component includes activation signals originating from atrial myocardial tissue whereas the far-field signal component includes activation signals originating from the ventricular myocardial tissue. The near-field activation signal component can be further analyzed to find the presence of a pathology and to determine a location suitable for ablation for treatment of the pathology, e.g., ablation therapy.

The processing system 32 includes dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired activation signals. In some embodiments, the processing system 32 includes a general purpose microprocessor and/or a specialized microprocessor (e.g., a digital signal processor, or DSP, which may be optimized for processing activation signals) that executes instructions to receive, analyze and display information associated with the received activation signals. In such implementations, the processing system 32 can include program instructions, which when executed, perform part of the signal processing. Program instructions can include, for example, firmware, microcode or application code that is executed by microprocessors or microcontrollers. The above-mentioned implementations are merely exemplary, and the reader will appreciate that the processing system 32 can take any suitable form.

In some embodiments, the processing system 32 may be configured to measure the intrinsic electrical activity in the myocardial tissue adjacent to the electrodes 24. For example, in some embodiments, the processing system 32 is configured to detect intrinsic electrical activity associated with a dominant rotor in the anatomical feature being mapped. Studies have shown that dominant rotors have a role in the initiation and maintenance of atrial fibrillation, and ablation of the rotor path and/or rotor core may be effective in terminating the atrial fibrillation. In either situation, the processing system 32 processes the sensed activation signals to isolate the near-field signal component and generate an APD map based on the isolated near-field signal component. The APD map may be used by the physician to identify a site suitable for ablation therapy.

The ablation probe 16 includes a flexible catheter body 34 that carries one or more ablation electrodes 36. The one or more ablation electrodes 36 are electrically connected to a radio frequency generator (RF) 37 that is configured to deliver ablation energy to the one or more ablation electrodes 36. The ablation probe 16 is movable with respect to the anatomical feature to be treated, as well as the structure 20. The ablation probe 16 is positionable between or adjacent to electrodes 24 of the structure 20 as the one or more ablation electrodes 36 are positioned with respect to the tissue to be treated.

The processing system 32 outputs to a device 40 the generated APD map for viewing by a physician. In the illustrated embodiment, device 40 is a CRT, LED, or other type of display, or a printer). The device 40 presents the APD map in a format most useful In the physician. In addition, the processing system 32 may generate position-identifying output for display on the device 40 that aids the physician in guiding the ablation electrode(s) 36 into contact with tissue at the site identified for ablation.

FIG. 2 illustrates an embodiment of the mapping catheter 14 including electrodes 24 at the distal end suitable for use in the system 10 shown in FIG. 1. The mapping catheter 14 has a flexible catheter body 18, the distal end of which carries the three dimensional structure 20 configured to carry the mapping electrodes or sensors 24. The mapping electrodes 24 sense intrinsic electrical activity, e.g., activation signals, in the myocardial tissue, the sensed activity is then processed by the processing system 32 to assist the physician in identifying the site or sites having a heart rhythm disorder or other myocardial pathology via a generated and displayed APD map. This process is commonly referred to as mapping. This information can then be used to determine an appropriate location for applying appropriate therapy, such as ablation, to the identified sites, and to navigate the one or more ablation electrodes 36 to the identified sites.

The illustrated three-dimensional structure 20 comprises a base member 41 and an end cap 42 between which flexible splines 44 generally extend in a circumferentially spaced relationship. As discussed above, the three dimensional structure 20 takes the form of a basket defining an open interior space 22. In some embodiments, the splines 44 are made of a resilient inert material, such as Nitinol metal or silicone rubber, and are connected between the base member 41 and the end cap 42 in a resilient, pretensed condition, to bend and conform to the tissue surface they contact. In the illustrated embodiment, eight splines 44 form the three dimensional structure 20. Additional or fewer splines 44 could be used in other embodiments. As illustrated, each spline 44 carries eight mapping electrodes 24. Additional or fewer mapping electrodes 24 could be disposed on each spline 44 in other embodiments of the three dimensional structure 20. In the illustrated embodiment, the three dimensional structure 20 is relatively small (e.g., 40 mm or less in diameter). In alternative embodiments, the three dimensional structure 20 is even smaller or larger (e.g., 40 mm in diameter or greater).

A slidable sheath 50 is movable along the major axis of the catheter body 18. Moving the sheath 50 forward (i.e., toward the distal end) causes the sheath 50 to move over the three dimensional structure 20, thereby collapsing the structure 20 into a compact, low profile condition suitable for introduction into and/or removal from an interior space of an anatomical structure, such as, for example, the heart. In contrast, moving the sheath 50 rearward (i.e., toward the proximal end) exposes the three dimensional structure 20, allowing the structure 20 to elastically expand and assume the pretensed position illustrated in FIG. 2. Further details of embodiments of the three dimensional structure 20 are disclosed in U.S. Pat. No. 5,647,870, entitled “Multiple Electrode Support Structures,” which is hereby expressly incorporated herein by reference in its entirety.

A signal wire (not shown) is electrically coupled to each mapping electrode 24. The wires extend through the body 18 of the mapping catheter 20 into a handle 54, in which they are coupled to an external connector 56, which may be a multiple pin connector. The connector 56 electrically couples the mapping electrodes 24 to the processing system 32. Further details on mapping systems and methods for processing signals generated by the mapping catheter are discussed in U.S. Pat. No. 6,070,094, entitled “Systems and Methods for Guiding Movable Electrode Elements within Multiple-Electrode Structure,” U.S. Pat. No. 6,233,491, entitled “Cardiac Mapping and Ablation Systems,” and U.S. Pat. No. 6,735,465, entitled “Systems and Processes for Refining a Registered Map of a Body Cavity,” the disclosures of which are hereby expressly incorporated herein by reference.

It is noted that other multi-electrode structures could be deployed on the distal end of the mapping catheter 14. It is further noted that the multiple mapping electrodes 24 may be disposed on more than one structure rather than, for example, the single mapping catheter 14 illustrated in FIG. 2. For example, if mapping within the left atrium with multiple mapping structures, an arrangement comprising a coronary sinus catheter carrying multiple mapping electrodes and a basket catheter carrying multiple mapping electrodes positioned in the left atrium may be used. As another example, if mapping within the right atrium with multiple mapping structures, an arrangement comprising a decapolar catheter carrying multiple mapping electrodes for positioning in the coronary sinus, and a loop catheter carrying multiple mapping electrodes for positioning around the tricuspid annulus may be used.

Although the mapping electrodes 24 have been described as being carried by dedicated mapping probes, such as the mapping catheter 14, the mapping electrodes may be carried on non-mapping dedicated probes or multifunction probes. For example, an ablation catheter, such as the ablation catheter 16, can be configured to include one or more mapping electrodes 24 disposed on the distal end of the catheter body and coupled to the signal processing system 32 and guidance system 38. As another example, the ablation electrode at the distal end of the ablation catheter may be coupled to the signal processing system 32 to also operate as a mapping electrode.

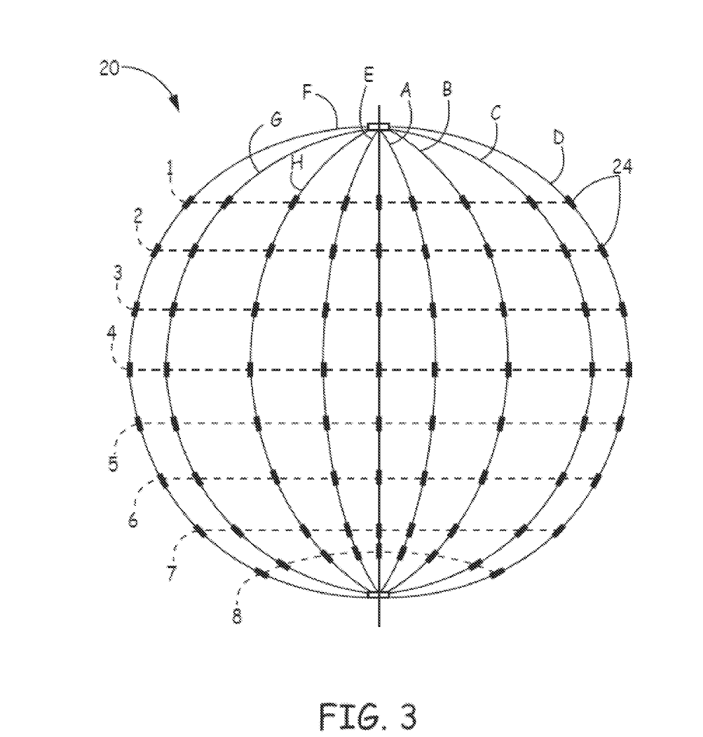

To illustrate the operation of the system 10, FIG. 3 is a schematic side view of an embodiment of the basket structure 20 including a plurality of mapping electrodes 24. In the illustrated embodiment, the basket structure includes 64 mapping electrodes 24. The mapping electrodes 24 are disposed in groups of eight electrodes (labeled 1, 2, 3, 4, 5, 6, 7, and 8) on each of eight splines (labeled A, B, C, D, E, F, G, and H). While an arrangement of sixty-four mapping electrodes 24 is shown disposed on a basket structure 20, the mapping electrodes 24 may alternatively be arranged in different numbers, on different structures, and/or in different positions. In addition, multiple basket structures can be deployed in the same or different anatomical structures to simultaneously obtain signals from different anatomical structures.

After the basket structure 20 is positioned adjacent to the anatomical structure to be treated (e.g., left atrium or left ventricle of the heart), the processing system 32 is configured to record the activation signals from each electrode 24 channel related to intrinsic physiological activity of the anatomical structure, Le. the electrodes 24 measure electrical activation signals intrinsic to the physiology of the anatomical structure.

The processing system 32 is further configured to identify substantially similar activation signals based on a self-correlation algorithm and identify which activation signals correspond to far-field activation signals. The processing system 32 bins the acquired activation signals according to a similarity threshold of the self-correlation algorithm. The threshold can be adjusted to increase or decrease the number of bins and thus increase or decrease the level similarity amongst activation signals in each bin. The processing system 32 blanks or filters out the bin or bins activations signals that are identified as far-field activations based on at least one of a subtraction between the activation signals and a template, a suppression of an amplitude for a duration of a beat, and zeroing the signal for a duration of the beat. The remaining activation signals are related to local activation signals which can then be used to generate activation maps of the anatomical structure.

In some embodiments, the processing system 32 determines a temporal window based on a far-field activation signal acquired with a far-field electrode. A far-field electrode, such as an ECG electrode, acquires an ECG signal which can be utilized as far-field activation signal. The far-field activation signals are aligned and a far-field activation temporal window is therefrom determined. The temporal window describes a window during which a far-field activation signal is most likely to occur. Local activation signals that fall within the temporal window may correspond to far-field activations. The processing system 32 identifies which bins of activation signals correspond to the determined temporal window and identifies the corresponding bins as including far-field activation signals. The processing system 32 in turn filters or blanks the beats corresponding to the identified bins, and the remaining activation signals can then be used to generate the activation maps.

In some embodiments, the processing system 32 generates a morphology template for each bin of activation signals which describes a morphology of the activation signals belonging to the specified bin. To generate the morphology template, the processing system 32 aligns the activation signals within each bin according to a dominant feature of the activations signals, e.g. an R-wave peak or the like. A morphology descriptor is determined from the aligned activation signals and the morphology template therefrom. The processing system 32 compares each generated morphology template to a generated or pre-defined characteristic template which defines a characteristic morphology of far-field activation signals. The processing system 32 identifies bins of activation signals that correspond to far-field activations based on the comparison of the morphology template and the characteristic morphology template. The processing system can blank or filter the beats corresponding to the identified bins and a resulting activation map can be generated based on the filtered or blanked activation signals.

In some embodiments, the processing system 32 generates a frequency template for each bin of activation signals which describes frequency components of the activation signals belonging to the specified bin. To generate the frequency template, the processing system 32 determines the frequency components, e.g., via a Fourier transform or the like, of each activation signal within a given bin. The frequency components are correlated to one another, e.g., via averaging or the like, to determine the frequency template for the corresponding bin. The processing system 32 compares each generated frequency template to a generated or pre-defined characteristic template which defines characteristic frequency components of far-field activation signals. For example, a far-field activation signal will, in general, have a larger low frequency component than a local signal and therefore the characteristic frequency template can be generated accordingly. The processing system 32 identifies bins of activation signals that correspond to far-field activations based on the comparison of each frequency template and the characteristic frequency template. The processing system can blank or filter the beats corresponding to the identified bins and a resulting activation map can be generated based on the filtered or blanked activation signals.

In some embodiments, the processing system 32 generates a temporal frequency template for each bin of activation signals which describes temporal frequency of the activation signals of a given bin. To generate the temporal frequency template, the processing system 32 calculates a duration between consecutive activation signals within a given bin and determines a mean, variance, or other metric regarding the duration between consecutive activations of the bin. The processing system 32 compares each generated temporal frequency template to a generated or pre-defined characteristic template which defines temporal frequency characteristic of far-field activation signals. For example, far-field activation signals will exhibit less temporal variability between consecutive activation signals and therefore the characteristic temporal frequency template can be generated accordingly. The processing system 32 identifies bins of activation signals that correspond to far-field activations based on the comparison of each temporal frequency template and the characteristic temporal frequency template. The processing system can blank or filter the beats corresponding to the identified bins and a resulting activation map can be generated based on the filtered or blanked activation signals.

The generated activation maps can be reviewed by a physician to identify and locate pathologies in the cardiac tissue such as arrhythmic disorders, e.g. a dominant rotor, rotor core, or rotor path. In some embodiments, the method can include identifying an anomaly or pathology at the anatomical location, The location of the pathology and the activation map of the anatomical structure can be displayed to the physician via the device 40. A therapy device, such as the ablation probe 16, can be deployed adjacent to the pathology at the targeted location and therapeutic energy can be applied to treat the pathology.

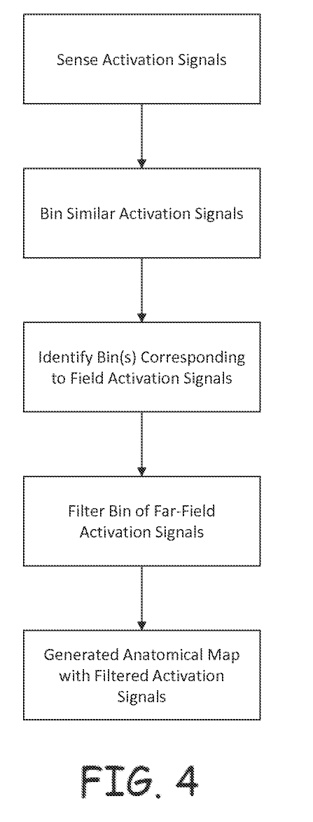

The system 10 is configured to perform a method of mapping an anatomical structure as illustrated in FIG. 4. After the mapping electrodes 24 or disposed in or adjacent to an anatomical structure, e.g. cardiac tissue, the system 10 senses electrical activation signals associated with intrinsic physiological activity of the anatomical structure. The system 10 bins substantially similar sensed activation signals according to a self-correlation algorithm. The self-correlation algorithm identifies patterns among the sensed activation signals and bins the activation signals into distinct bins according to a degree of similarity among the identified patterns. The system 10 is configured to identify at least one of the bin which corresponds to a far-field activation signal. A far-field activation signal can introduce noise or error when mapping the anatomical structure. By identifying the bin and/or bins of activation signals which correspond to far-field activation signals, the system 10 can filter the activation signals which correspond to the at least one identified bin of far-field activation signals from the sensed activation signals and generate an anatomical map or activation map of the anatomical structure based on the filtered activation signals.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method for mapping an anatomical structure, the method comprising:
sensing activation signals of intrinsic physiological activity with a plurality of electrodes disposed in or near the anatomical structure;
binning substantially similar sensed activation signals according to a self-correlation algorithm which identifies patterns among the sensed activation signals; and
identifying at least one bin which corresponds to a far-field activation signal.

2. The method according to claim 1, wherein the step of identifying at least one bin further includes:
acquiring a plurality of far-field activation signals with at least one far-field sensor;
aligning the far-field activation signals according to a characteristic feature;
determining a temporal window for a far-field activation signal; and
identifying at least one bin of activation signals which correspond to the temporal window as far-field activation signals.

3. The method according to claim 1, wherein the step of identifying at least one bin further includes:
generating a characteristic template for each bin based on a morphology of the corresponding activation signals;
generating a morphology template which identifies morphology of a far-field activation signal; and
identifying at least one bin of activation signals as far-field activation signals according to a comparison of each characteristic template and the morphology template.

4. The method according to claim 1, wherein the step of identifying at least one bin further includes:
generating a characteristic template for each bin based on a frequency component of the corresponding activation signals;
generating a frequency template which identifies frequency components of a far-field activation signal; and
identifying at least one bin of activation signals as far-field activation signals according to a comparison of each characteristic template and the frequency template.

5. The method according to claim 1, wherein the step of identifying at least one bin further includes:
generating a characteristic template for each bin based on a temporal frequency of the corresponding activation signals;
generating a temporal template which identifies a temporal frequency of a far-field activation signal; and
identifying at least one bin of activation signals as far-field activation signals according to a comparison of each characteristic template and the temporal template.

6. The method according to claim 1, further including:
filtering the activation signals which correspond to the at least one identified bin from the sensed activation signals.

7. The method according to claim 1, wherein the activation signals are filtered based on at least one of a subtraction between the activation signals and a template, a suppression of an amplitude for a duration of a beat, and zeroing the signal for a duration of the beat.

8. The method according to claim 6, further including:
generating an activation map of the anatomical structure based on the filtered activation signals.

9. A method for mapping an anatomical structure, the method comprising:
sensing activation signals of intrinsic physiological activity with a plurality of electrodes disposed in or near the anatomical structure;
binning substantially similar sensed activation signals according to a self-correlation algorithm which identifies patterns among the sensed activation signals;
identifying at least one bin which corresponds to a far-field activation signal;
filtering the activation signals which correspond to the at least one identified bin from the sensed activation signals; and
generating an activation map of the anatomical structure based on the filtered activation signals.

10. The method according to claim 8, wherein the step of identifying at least one bin further includes:
acquiring a plurality of far-field activation signals with at least one far-field sensor;
aligning the far-field activation signals according to a characteristic feature;
determining a temporal window for a far-field activation signal; and
identifying at least one bin of activation signals which correspond to the temporal window as far-field activation signals.

11. The method according to claim 8, wherein the step of identifying at least one bin further includes:
generating a characteristic template for each bin based on a morphology of the corresponding activation signals;
generating a morphology template which identifies a morphology of a far-field activation signal; and
identifying at least one bin of activation signals as far-field activation signals according to a comparison of each characteristic template and the morphology template.

12. The method according to claim 8, wherein the step of identifying at least one bin further includes:
generating a characteristic template for each bin based on a frequency component of the corresponding activation signals;
generating a frequency template which identifies frequency component of a far-field activation signal; and
identifying at least one bin of activation signals as far-field activation signals according to a comparison of each characteristic template and the frequency template.

13. The method according to claim 8, wherein the step of identifying at least one bin further includes:
generating a characteristic template for each bin based on a temporal frequency of the corresponding activation signals;
generating a temporal template which identifies a temporal frequency of a far-field activation signal; and
identifying at least one bin of activation signals as far-field activation signals according to a comparison of each characteristic template and the temporal template.

14. An anatomical mapping system comprising:
a plurality of mapping electrodes configured to detect activation signals of intrinsic physiological activity within an anatomical structure, each of the plurality of mapping electrodes having an electrode location and channel;
a processing system associated with the plurality of mapping electrodes, the processing system configured to record the detected activation signals and associate at least one of the plurality of mapping electrodes with each recorded activation signal, the processing system further configured to bin substantially similar sensed activation signals according to a self-correlation algorithm which identifies patterns among the sensed activation signals, and identify at least one bin which corresponds to a far-field activation signal.

15. The anatomical mapping system according to claim 13, wherein the processing system is further configured to filter the activation signals which correspond to the at least one identified bin from the sensed activation signals, and generate an activation map of the anatomical structure based on the filtered activation signals.

16. The anatomical mapping system according to claim 14, further including:
a display configured to display the activation map of the filtered activation signals.

17. The anatomical mapping system according to claim 13, wherein, to identify at least one bin which corresponds to a far-field activation signal, the processing system is further configured to align a plurality of far-field activation signals acquired with at least one far-field sensor according to a characteristic feature, determining a temporal window for a far-field activation signal, and identify at least one bin of activation signals which correspond to the temporal window as far-field activation signals.

18. The anatomical mapping system according to claim 13, wherein, to identify at least one bin which corresponds to a far-field activation signal, the processing system is further configured to generate a characteristic template for each bin based on a morphology of the corresponding activation signals, generate a morphology template which identifies a morphology of a far-field activation signal, and identify at least one bin of activation signals as far-field activation signals according to a comparison of each characteristic template and the morphology template.

19. The anatomical mapping system according to claim 13, wherein, to identify at least one bin which corresponds to a far-field activation signal, the processing system is further configured to generate a characteristic template for each bin based on a frequency component of the corresponding activation signals, generate a frequency template which identifies frequency component of a far-field activation signal, and identify at least one bin of activation signals as far-field activation signals according to a comparison of each characteristic template and the frequency template.
